(19) United States
(12) Patent Application Publication
(10) Pub. No.: US 2014/0349982 A1
(43) Pub. Date: Nov. 27, 2014

(54) METHODS OF TREATING ULCERATIVE COLITIS

(71) Applicant: Salix Pharmaceuticals, Inc., Raleigh, NC (US)

(72) Inventors: William Forbes, Raleigh, NC (US); Enoch Bortey, Chapel Hill, NC (US); Craig Paterson, Durham, NC (US); Pam Golden, Durham, NC (US)

(73) Assignee: Salix Pharmaceuticals, Inc., Raleigh, NC (US)

(21) Appl. No.: 14/282,888

(22) Filed: May 20, 2014

Related U.S. Application Data
(60) Provisional application No. 61/825,929, filed on May 21, 2013.
(60) Provisional application No. 61/905,015, filed on Nov. 15, 2013.
(60) Provisional application No. 61/986,075, filed on Apr. 29, 2014.

(51) Int. Cl. A61K 31/58; A61J 1/14; A61M 39/22; A61K 9/00

(52) U.S. Cl. CPC A61K 31/58; A61K 9/0031; A61J 1/1412; A61M 39/22
USPC 514/174; 604/514

(57) ABSTRACT

Provided herein are methods of treating and inducing ulcerative colitis in a subject. Also provided are methods of treating subjects with mild to moderate active ulcerative colitis, including ulcerative proctitis and proctosigmoiditis. Also provided are methods of administering budesonide to a subject to treat ulcerative colitis, including ulcerative proctitis and proctosigmoiditis.

### CROSS REFERENCE TO RELATED APPLICATIONS

This application claims the benefit of priority to U.S. Provisional Application 61/825,929, filed May 21, 2013, U.S. Provisional Application 61/905,015, filed Nov. 15, 2013, and U.S. Provisional Application 61/986,075, filed Apr. 29, 2014, which are herein incorporated by reference in their entirety.

### BACKGROUND

Ulcerative colitis (UC) is an idiopathic, chronic relapsing/remitting, non-specific inflammatory disease of the colonic mucosa. The disease is characterized by recurring episodes of inflammation primarily involving the mucosal layer and occasionally the submucosa of the colon. Acute episodes are characterized by chronic diarrhea or constipation, rectal bleeding, cramping and abdominal pain. Disease progression may be associated with urgency to defecate, tenesmus, anemia, and hypoalbuminemia. Systemic manifestations may include anorexia, weight loss, fatigue, fever, increased sedimentation rate, arthritis, eye inflammation, anxiety, tachycardia, and elevated liver function tests (LFTs). Criteria used to diagnose UC include clinical assessment, endoscopic evaluation, and stool sample and histological grading.

Despite improved understanding over the past several decades of the genetics, environmental factors, and inflammatory mechanisms involved in UC, the etiology and pathogenesis of the disease remain undefined.

Historically, ulcerative proctitis (UP) has been categorized as a milder form of UC. Reported rates of UP range from 25 to 55% of all UC cases at initial diagnosis (Meucci et al. 2000. Am J Gastroenterol 95(2):469-473). Ulcerative disease confined to the rectum and sigmoid colon is characterized as ulcerative proctosigmoiditis (UPS). Symptoms characteristic of UP/UPS include rectal bleeding, urgency, tenesmus, diarrhea or constipation, and rectal pain.

Primary therapies for distal UC include use of rectal and/or oral drugs from the aminosalicylate class (e.g., mesalamine and sulfasalazine), or corticosteroids (e.g., prednisone, betamethasone, or methylprednisolone), depending on severity of the episode. Alternative and more experimental agents include immunosuppressive agents (azathioprine, 6-mercaptopurine, cyclosporine, and methotrexate), 5-lipoxygenase selective inhibitors, topical short chain fatty acids, biologics (e.g., infliximab, etanercept, adalimumab) and bismuth subsalicylate enemas. Extensive colitis and pancolitis are currently treated either orally or intravenously, with or without concomitant rectal administration. However, discomfort or anal irritation from the suppositories leads to lack of tolerance of topical therapy in some cases.

Ulcerative colitis patients with UP/UPS are very difficult to treat and often present as the most challenging subset of patients given the limitations of current treatment options. For patients with distal disease, treatment with approved oral UC agents is often ineffective due to insufficient distribution of active drug to the distal colon. To date, only hydrocortisone foam/enema and mesalamine rectal suspension enema/suppositories are approved by the Food and Drug Administration (FDA) for treatment of distal UC, and only enema formulations are indicated for patients with disease extending beyond the rectum. Patients with distal UC have tremendous difficulty retaining enemas due to high volume and consistency of the formulation over recommended retention periods suggested in labeling (˜8 hours). In addition, patients can experience tenesmus, and the use of enemas can be associated with pain and negative effect on quality of life. There remains an unmet medical need for safe and effective therapies in the treatment of distal UC (UP/UPS) that overcome limitations of existing products. There is also a need to provide alternative methods of treating UP or UPS that are safe, effective and, in the case of administration with a steroid-class agent, provide minimal steroid-like side effects.

### SUMMARY OF THE INVENTION

Described herein are methods of treating ulcerative colitis in a subject. The methods includes administering a foam composition comprising budesonide to the subject, wherein the subject is administered 2 mg budesonide BID for two weeks, followed by 2 mg budesonide QD for four weeks. In some embodiments, the composition is administered rectally.

In some embodiments, the method includes administering a foam composition comprising budesonide to the subject, wherein the subject is administered 2 mg/25 mL budesonide foam BID for 2 weeks followed by 2 mg/25 mL budesonide foam QD for 4 weeks. In some embodiments, the method includes administering a foam composition comprising budesonide to the subject, wherein the subject is administered 2 mg/10-40 mL budesonide foam BID for 2 weeks followed by 2 mg/10-40 mL budesonide foam QD for 4 weeks.

In some embodiments, the subject suffers from active mild to moderate ulcerative proctitis and/or ulcerative proctosigmoiditis. In some embodiments, the subject suffers from one or more symptoms selected from the group of: rectal bleeding, urgency, tenesmus, diarrhea, constipation and rectal pain.

Further embodiments are directed to methods of inducing remission in subjects with active mild to moderate distal ulcerative colitis (UC) comprising, administering a foam composition comprising budesonide to the subject, wherein the subject is administered 2 mg budesonide twice daily (BID) for two weeks, followed by 2 mg budesonide once daily (QD) for four weeks.

In some embodiments, the disease extends from about 5 cm to about 40 cm from the anal verge of the subject. For example, in some embodiments, the disease extends about from about 1 cm to about 5 cm from the anal verge of the subject. In another embodiment, the disease extends from about 5 cm to about 15 cm from the anal verge of the subject, in other embodiments the disease extends to about 15 cm from the anal verge of the subject.

In some embodiments, the disease extends from 5 cm to about 40 cm from the anal verge of the subject, in another embodiment, the disease extends from 15 cm to about 40 cm from the anal verge of the subject, in another embodiment, the disease extends up to about 40 cm from the anal verge of the subject.

Further embodiments are directed to methods of inducing remission in subjects with active mild to moderate distal ulcerative colitis (UC) extending up to 40 cm from the anal verge comprising administering a foam composition comprising budesonide to the subject, wherein the subject is administered 2 mg budesonide twice daily (BID) for two weeks, followed by 2 mg budesonide once daily (QD) for four weeks.

In some embodiments, the subject exhibits histological changes characteristic of ulcerative colitis, ulcerative proctitis and/or ulcerative proctosigmoiditis. In some embodiments, the subject exhibits a Modified Mayo Disease Activity Index (MMDAI) score of between about 5 and 10 prior to administration of the composition. In some embodiments, the subject exhibits a score of ≧2 on the MMDAI rectal bleeding component prior to administration of the composition. In some embodiments, the subject exhibits a score of ≧2 on the MMDAI endoscopy or sigmoidoscopy component prior to administration of the composition.

In some embodiments, administration of the composition results in one or more selected from the group of: an endoscopy score of ≦1, a rectal bleeding score of 0, and an improvement or no change from baseline in stool frequency subscales of the Modified Mayo Disease Activity Index (MMDAI).

In some embodiments, administration of the composition results in one or more selected from the group of: an endoscopy score of ≦1, a rectal bleeding score of 0, and an improvement or no change from baseline in stool frequency subscales of the Modified Mayo Disease Activity Index (MMDAI). For example, in some embodiments, an improvement in stool frequency can include a combined score of ≦2 for bowel frequency and physician's global assessment in the MMDAI subscales.

In some embodiments, administration of the composition results in an MMDAI rectal bleeding score of 0. In some embodiments, administration of the composition results in an MMDAI endoscopy score of 0 or 1.

In some embodiments, administration of the composition results in an MMDAI overall of ≦2. For example, in some embodiments, administration of the composition results in an MMDAI overall of ≦1.

In some embodiments, administration of the composition results in an improvement of ≧1 point from baseline in the MMDAI endoscopy score. In some embodiments, administration of the composition results in an improvement of ≧1 point from baseline in the MMDAI rectal bleeding score.

In some embodiments, administration of the composition results in an improvement of ≧3 points from baseline in the MMDAI total score, including a 1 point improvement in both rectal bleeding and endoscopy scores.

In any of the foregoing embodiments, an improvement in disease symptoms and/or progress can be observed for up to 6 weeks after administration of the composition commences. For example, in some embodiments, an improvement in disease symptoms and/or progress can be observed for up to 4 weeks after administration of the composition commences.

In any of the foregoing embodiments, the incidence of headaches is lower than in subjects administered 2 or 4 mg budesonide foam BID for 6 weeks or 2 mg QD for 8 weeks. In any of the foregoing embodiments, the incidence of headaches is lower than in subjects administered 2 or 4 mg budesonide foam BID for 6 weeks or 2 mg QD for 8 weeks, wherein about 2% of subjects administered 2 mg/10-40 mL budesonide foam BID for 2 weeks followed by 2 mg/10-40 mL budesonide foam QD for 4 weeks have headaches. In any of the foregoing embodiments, the incidence of headaches is lower than in subjects administered 2 or 4 mg budesonide foam BID for 6 weeks or 2 mg QD for 8 weeks, wherein from about 1-2.9% of subjects administered 2 mg/10-40 mL budesonide foam BID for 2 weeks followed by 2 mg/10-40 mL budesonide foam QD for 4 weeks have headaches.

In a further embodiment, approximately 2% of the subjects experience headaches.

In another embodiment, approximately 1.5% of the subjects experience headaches.

In a further embodiment, less than 3% of the subjects experience headaches.

In another embodiment, approximately 1.5-3% of the subjects experience headaches. In another embodiment, approximately 1.5-2.9% of the subjects experience headaches.

In any of the foregoing embodiments, the incidence of nervous system disorders can be lower than in subjects administered budesonide foam 2 mg QD for 8 weeks.

In some embodiments, the incidence of nervous system disorders is lower than in subjects administered budesonide foam 2 mg QD for 4 weeks.

In any of the foregoing embodiments, the incidence of respiratory side effect can be lower than in subjects administered budesonide foam 2 or 4 mg budesonide foam BID for 6 weeks or 2 mg QD for 4 or 8 weeks.

In another embodiment, 0% of the subjects experience respiratory adverse events.

In any of the foregoing embodiments, the incidence of gastrointestinal side effects can be lower than in subjects administered budesonide foam 2 mg QD for 4 weeks.

Provided herein are methods of alleviating symptoms in a subject with ulcerative colitis, comprising administering a foam composition comprising budesonide to the subject, wherein the subject is administered 2 mg budesonide BID for two weeks, followed by 2 mg budesonide QD for four weeks.

In one embodiment, the symptoms are selected from the group consisting of diarrhea, constipation, urgency, tenesmus, rectal bleeding, rectal pain, cramping and abdominal pain.

Provided herein are methods of treating ulcerative colitis, comprising administering a foam composition comprising budesonide to the subject, wherein the subject is administered 2 mg budesonide BID for two weeks, followed by 2 mg budesonide QD for four weeks, wherein subjects experience a lower than expected systemic level of budesonide in the four weeks of QD dosing.

In one embodiment, the systemic exposure of budesonide foam is affected by the severity of the disease state.

In another embodiment, there is a decrease in the elimination rate constant associated with an increase in the severity of disease symptoms.

In one embodiment, an increased age in a subject correlates to a decrease in the systemic elimination rate of budesonide.

In another embodiment, the age of a subject leads to a decrease in the elimination rate constant of budesonide.

In another embodiment, the age of a subject leads to a 0.1 decrease in the elimination rate constant of budesonide.

In another embodiment, the age of a subject leads to a 0.2 decrease in the elimination rate constant of budesonide.

In another embodiment, the age of a subject leads to a 0.3 decrease in the elimination rate constant of budesonide.

In another embodiment, the age of a subject leads to a 0.4 decrease in the elimination rate constant of budesonide.

In another embodiment, the age of a subject leads to a 0.5 decrease in the elimination rate constant of budesonide.

In other embodiments, a subject has an increase in the efficacy of budesonide despite a decrease in systemic concentration of budesonide in weeks 3-6.

In a further embodiment, estimates of AUC for plasma budesonide are correlated with increased sensitivity to ACTH.

In another embodiment, there is a gradual reduction in systemic exposure to budesonide.

In some embodiments, the budesonide solution is used to produce rectal foams. For example, a rectal foam product may be contained in a delivery device with a closure system for the foam product comprising a canister and, a metering valve.

In another embodiment, the metering valve may comprise a stem.

In a further embodiment, the delivery device may further comprise a safety tab to, for example, to prevent accidental actuation. In certain embodiments, the tab could be removed prior to use.

In a further embodiment, the device delivers a dose when only when it is held inverted.

In a further embodiment, the device delivers a dose when it is held in any direction.

In another embodiment, the device delivers a dose only when inverted.

Other embodiments are disclosed infra.

### DESCRIPTION OF THE DRAWINGS

FIG. 1 is a bar chart indicating the percentage of subjects who achieved remission in the treatment and placebo groups in Study 1, Study 2 and combined Study 1 and Study 2 data, described herein. Studies 1 and 2 are two identical randomized double-blind, placebo-controlled studies of subjects.

FIG. 2 is a bar chart indicating the mean plasma budesonide concentrations in subjects with different dosing regimens.

FIG. 3 is a graph indicating the mean and standard deviation of morning cortisol levels in subjects.

### DETAILED DESCRIPTION

The discoveries described herein result, in-part, from two identical randomized double-blind, placebo-controlled study of patients who present with mild to moderate active ulcerative colitis. Corticosteroid use is a mainstay for the treatment of active inflammatory bowel disease (IBD); however, use of these agents is limited by an associated side effect profile following systemic delivery attributed to many drugs within the class, which includes cosmetic (e.g., acne, moonface) and clinically significant effects (e.g., psychological, hypertension, dyspepsia, impaired glucose tolerance (IGT)). Furthermore, significant risks of long-term corticosteroid use, such as osteoporosis, osteonecrosis, cataracts, and Type 2 diabetes mellitus (T2DM) can preclude long term use.

Budesonide is a potent synthetic glucocorticoid that possesses anti-inflammatory, anti-allergic, anti-exudative and anti-edematic properties. It was first developed for the treatment of bronchial asthma and rhinitis and later in the mid 1980's, for the treatment of inflammatory bowel disease (IBD). The relatively high water solubility of budesonide allows for rapid dissolution, facilitating rapid transport to the bowel wall and high uptake into tissue, producing high concentrations and high activity in target tissues when applied topically. Even though budesonide exhibits high potency at the local application site, it possesses minimal systemic bioavailability and thus produces reduced steroid-like side effects as compared to other agents in its class. Recent clinical studies evaluating oral and rectal preparations of budesonide have demonstrated that this agent elicits less suppression of serum cortisol levels than observed with other glucocorticoids (e.g., methylprednisolone, prednisolone or hydrocortisone), thus resulting in minimal to no HPA suppression, with subsequent reduction of steroid-like adverse effects when compared to other rectally administered corticoid agents. The estimates of AUC for plasma budesonide were found to be correlated with increased sensitivity to ACTH at the 42 day ACTH stimulation test. This increase in sensitivity suggests that the exposure seen with 2 mg QD budesonide rectal foam are very unlikely to result in reduced sensitivity of the HPA axis. Rather, systemic exposure to budesonide predicted increased responsiveness in the ACTH stimulation test.

As described herein, mild to moderate active ulcerative colitis, including ulcerative proctitis (UP) and proctosigmoiditis (UPS) were treated in the two studies, Study 1 and Study 2, with rectally-administered budesonide foam (2 mg budesonide/25 mL foam) twice daily (BID) for two weeks, followed by rectally-administered budesonide foam treatment once daily (QD) for four weeks. The results indicate that the dosing regimen of budesonide foam was surprisingly effective in treating UP and UPS. These results were unexpected given that rectally-administered 5-ASA enemas were identified as providing superior results relative to hydrocortisone enemas in the treatment of mild to moderately active ulcerative colitis in patients (Campieri et al. 1981. “Treatment of Ulcerative Colitis with High-Dose 5-Aminosalicylic Acid Enemas.” The Lancet 2(8241):270-272). Marshall and Irvine also identified the efficacy of rectal 5-ASA therapy in the treatment of mild to moderately active ulcerative colitis in a meta-analysis (Marshall, J. K. and E. J. Irvine. 1995. “Rectal aminosalicylate therapy for distal ulcerative colitis: A meta-analysis.” Aliment Pharmcol Ther 9(3):293-300).

Furthermore, administration of budesonide foam at 4 mg per day (2 mg BID budesonide) did not show significant improvement in treating mild to moderately active ulcerative colitis compared to administration of budesonide foam at 2 mg per day (2 mg QD budesonide) (unpublished). Accordingly, the regimen and timing of the budesonide foam treatment (2 mg BID for two weeks, followed by 2 mg QD for four weeks) provided surprisingly good clinical and statistically significant results with respect to previously-administered budesonide dosing regimens. In particular, less active ingredient is administered over the course of the dosing regimen described herein relative to previously administered dosing regimens. Better efficacy from less administered drug increases the safety profile of the drug, as well. For example, the methods described herein (for example, administering 2 mg budesonide foam BID for 2 weeks followed by administering 2 mg budesonide foam QD for 4 weeks) when comparing complete remission scores are between 7.3 to 13% more efficacious in 2 mg BID for 6 weeks; and are between 7.3 to 23% efficacious than 2 mg QD for 8 weeks. The methods of treating provided herein provide significantly less budesonide exposure to the subject than previous dosing methods. Methods of treatment include the gradual reduction in systemic exposure to budesonide.

Accordingly, disclosed herein are methods of treating ulcerative colitis (UC), including, for example, ulcerative proctitis (UP) or ulcerative proctosigmoiditis (UPS) with compositions comprising budesonide.

Budesonide is a high potency corticosteroid that was developed to minimize the systemic adverse consequences of first generation corticosteroids (e.g., hydrocortisone); and the foam formulation, described herein, for rectal administration was designed to improve both the subject's ability to retain the drug in the rectum following administration as well as to improve distribution of the active drug to the rectum and sigmoid colon.

Budesonide 2 mg rectal foam was highly effective in the treatment of UP/UPS in the two large studies, described herein. The budesonide foam formulation has demonstrated improved reach (e.g., spread) and rapid distribution of budesonide to the sigmoid colon and the rectum, without the pain and inconvenience associated with retention of enema formulations. The foam also provides more immediate and targeted therapy for distal UC than is available with oral therapies.

The improved reach of the budesonide formulation was demonstrated with scintigraphy studies in patients with distal UC, which demonstrated that budesonide foam distributed proximally up to 40 cm from the anal verge and reached the sigmoid colon in all patients.

Budesonide exhibited a surprisingly favorable safety profile in these studies described herein compared to other studies using budesonide foam in different treatment methods. By comparison to other corticosteroids, budesonide has minimal systemic exposure, which is further reduced by the ability to taper from a 2-week BID dose to a 4-week QD dose, reducing the potential for systemic steroid side effects. In contrast with other corticosteroid products for the treatment of UC, budesonide rectal foam appears to have a lower incidence of clinically significant adrenal suppression as measured by adrenocorticotropin hormone (ACTH) challenge and the adverse reaction profile. Reasons for this may include the lower systemic exposure and lower mineralocorticoid activity of budesonide foam.

Embodiments are directed to methods of treating active mild to moderate ulcerative colitis, including, for example, ulcerative proctitis and/or ulcerative proctosigmoiditis, comprising administering a composition comprising budesonide to a subject in need thereof. In some embodiments, the subject suffers from active, mild to moderate ulcerative proctitis and/or ulcerative proctosigmoiditis with disease extending from about 1 cm to about 45 cm from the anal verge. In some embodiments, confirmation of diagnosis is provided by endoscopy (e.g. colonoscopy or sigmoidoscopy).

In some embodiments, the subject suffers from symptoms of active, mild to moderate UC, including for example, UP and/or UPS, wherein the symptom is at least one selected from the group of: rectal bleeding, urgency, tenesmus, diarrhea, constipation and rectal pain.

In some embodiments, the subject is diagnosed with active, mild to moderate ulcerative colitis, including, for example, ulcerative proctitis and/or ulcerative proctosigmoiditis based on at least one criteria selected from the group of: histological changes characteristic of UP/UPS, a Modified Mayo Disease Activity Index (MMDAI) score of between about 5 and 10, a score of ≧2 on the MMDAI rectal bleeding component and a score of ≧2 on the MMDAI endoscopy or sigmoidoscopy component.

In some embodiments, the subject is diagnosed with active, mild to moderate ulcerative colitis, including, for example, ulcerative proctitis with disease limited to the rectum (e.g., extending up to about 15 cm relative to the anal verge). For example, the subject may have disease extending from about 1 cm, 2 cm, 3 cm, 4 cm, 5 cm, 6 cm, 7 cm, 8, cm, 9 cm, 10 cm, 11 cm, 12 cm, 13 cm, 14 cm, 15 cm, or from 0-15 cm or from 0.1-15 cm relative to the anal verge, or any distance in between.

In some embodiments, the subject is diagnosed with active, mild to moderate ulcerative colitis, including, for example, ulcerative proctosigmoiditis with disease limited to the rectum and sigmoid colon (e.g., extending up to about 40 cm relative to the anal verge). For example, the subject may have disease extending from about 1 cm, 2 cm, 3 cm, 4 cm, 5 cm, 6 cm, 7 cm, 8, cm, 9 cm, 10 cm, 11 cm, 12 cm, 13 cm, 14 cm, 15 cm, 16 cm, 17 cm, 18 cm, 19 cm, 20 cm, 21 cm, 22 cm, 23 cm, 24 cm, 25 cm, 26 cm, 27 cm, 28 cm, 29 cm, 30 cm, 31 cm, 32 cm, 33 cm, 34 cm, 35 cm, 36 cm, 37 cm, 38 cm, 39 cm, 40 cm, or from 0-40 cm or from 0.1-45 cm relative to the anal verge, or any distance in between.

In some embodiments, the subject is diagnosed with active, mild to moderate ulcerative proctosigmoiditis with disease extending up to about 45 cm relative to the anal verge. For example, the subject may have disease extending from about 1 cm, 2 cm, 3 cm, 4 cm, 5 cm, 6 cm, 7 cm, 8, cm, 9 cm, 10 cm, 11 cm, 12 cm, 13 cm, 14 cm, 15 cm, 16 cm, 17 cm, 18 cm, 19 cm, 20 cm, 21 cm, 22 cm, 23 cm, 24 cm, 25 cm, 26 cm, 27 cm, 28 cm, 29 cm, 30 cm, 31 cm, 32 cm, 33 cm, 34 cm, 35 cm, 36 cm, 37 cm, 38 cm, 39 cm, 40 cm, 41 cm, 42 cm, 43 cm, 44 cm, 45 cm or from 0-45 cm or from 0.1-45 cm relative to the anal verge, or any distance in between.

In some embodiments, the subject experiences no loss of responsiveness and/or did not become refractory to response. Despite a decrease in the systemic concentration of budesonide, subjects did not experience a decrease in the effect of budesonide over the course of treatment, as described herein. Subjects experienced an increase in the efficacy of budesonide despite a decrease in the subject's systemic concentration of budesonide. In other words, despite decreasing the dose over the course of administration and despite a reduction in systemic levels, a reduction that is greater than would be expected from the dose reduction, there is no loss in efficacy. This is a surprising result. Without wishing to be bound by any particular scientific theories, this could be due to an induction of metabolism local in the intestine. Thus, reducing systemic concentrations and not reducing efficacy because the efficacy may be driven by local concentrations of budesonide. This further demonstrates how the method of treatment and administration disclosed herein increases the safety profile of the budesonide foam.

### Compositions and Products

In some embodiments, budesonide is provided in a pharmaceutical composition. When budesonide is formulated, carriers and excipients such as, for example, lactose, microcrystalline cellulose, starch and anhydrous silica, lubricants such as, for example, hydrated castor oil, magnesium stearate, sodium lauryl sulfate and talc as well as binders such as, for example, starch, glucose, gum arabicum and mannitol, are used. If the composition is provided in a liquid state (e.g., to be foamed out of an applicator), liquid canisters or carriers can be used.

In some embodiments, the composition is contained in a canister with a metering valve system. In a further embodiment, the canister may be an aluminum pressurized container. The canister may be internally coated with lacquers and resins. The canister may optionally be supplied with or adopted to be used with or further comprise applicators for the administration of the liquid or foam. The applicators may be, for example, PVC, and may be, for example, coated with soft paraffin or liquid paraffin. Bags may also be provided for the disposal of used applicators.

The daily dose of budesonide is about 0.5 mg to 100 mg per day depending on the severity of the disease to be treated, the stage of the disease to be treated, any further diseases the patient may have, the age of the subject, the administration route and additional parameters which are known to the skilled person. In some embodiments, a daily dose of about 1 mg to 50 mg is provided. In some embodiments, a daily dose of about 5 mg to 20 mg is provided. The daily dose can be administered at one time per day or divided over the course of the day, for example, three times a day. In some embodiments, the pharmaceutical composition containing budesonide comprises between about 0.5 to 20 mg or between about 1 mg to 5 mg budesonide per unit dosage form.

The severity of the disease symptoms relates to the efficacy of the budesonide dosing regimen. The results of the two studies as well as their pooled data indicate that the systemic exposure of budesonide foam is different depending on the severity of the disease state. The studies revealed that there is a decrease in the elimination rate constant associated with an increase in the severity of disease symptoms. This again shows the surprising efficacy and safety provided by the methods of treatment and administration described herein.

Maximum plasma concentrations following administration of budesonide rectal foam were similar to those reported in healthy subjects for an extended-release oral formulation of budesonide (budesonide MMX) that was recently approved by the United States Food and Drug Administration (US FDA) for the induction of remission of UC. However, budesonide MMX had a substantially higher steady-state AUC (16.43 ng.h/mL) compared with budesonide rectal foam in healthy subjects (4.30 ng.h/mL) or UP/UPS patients (4.31 ng.h/mL, respectively, BUF-7/BIO; budesonide foam population pharmacokinetics report). This difference is attributable to the longer t½ observed for budesonide MMX versus budesonide rectal foam (8.2 h versus 4.0 h, respectively).

As the severity of the disease increases, there is a decrease in the elimination rate constant of budesonide. Subjects experiencing increased severity or symptoms of the disease exhibited decreased budesonide clearance. Surprisingly, the dosing regimen of budesonide proved more efficacious in subjects experiencing more severe symptoms. Subjects in the studies each had a confirmed diagnosis of active, mild to moderate UP or UPS. Severity of symptoms ranged from subjects with disease extending at least 5 cm to about 30 cm from the anal verge. A statistically significant effect was demonstrated between symptomatic severity and the elimination rate constant; with more severe symptoms exhibiting a lower rate of elimination.

It was also surprisingly found that there is an effect of age on the dosing regimen. Increased age in a subject correlates to a decrease in the systemic elimination rate of budesonide. Analysis on the effects of subjects between the ages of 18 and 75 years demonstrated that older subjects exhibit longer plasma residence time of budesonide while younger subjects experience decreased systemic exposure as compared to older subjects. While subjects with severe renal or hepatic impairment were not enrolled in this study, population pharmacokinetic modeling demonstrated that there was no significant effect of renal function (as measured by calculated creatinine clearance) or hepatic function (as measured by bilirubin, AST, or ALT) on the pharmacokinetics of budesonide foam administration. This is a surprising safety benefit of the budesonide foam dosing regimen described herein.

In some embodiments, a pharmaceutical composition comprising budesonide is provided, wherein the composition has a pH of about 1 to 6. For example, the pH of the composition can be about 1, 1.5, 2, 2.5, 3, 3.5, 4, 4.5, 5, 5.5, 6 or any value in between 0.1 and 6 pH. In some embodiments, the composition has a pH of about 3.5 for use as a rectal foam.

Any pharmaceutically acceptable organic and inorganic acids can be used to adjust the pH, for example hydrochloric acid, phosphoric acid, citric acid or tartaric acid.

In some embodiments, the pharmaceutical composition comprising budesonide is prepared as a concentrated budesonide solution with a pH of about 2 to 5, for example, as a concentrated solution with a pH of about 3.5 If it is necessary for subsequent use to adjust the pH to a physiologically tolerated value ≧3.5, this can take place shortly before use. This can happen, for example, by dilution or by addition of a base. The dilution process then increases the pH.

In some embodiments, a pharmaceutical composition comprising budesonide ulcerative co is provided, wherein the composition has a pH of about 6.0 or below. In some embodiments, the composition has a pH range of between about 3.5 and 5.0. In some embodiments, the composition has a pH range between about 4 and 4.5.

In some embodiments, the pharmaceutical composition comprising budesonide can be provided as a solution containing sodium EDTA (sodium ethylenediaminetetraacetic acid; Komplexon), which can increases the stability of the preparation.

In some embodiments, the pharmaceutical composition comprising budesonide can be provided as a solution containing cyclodextrins, such as, for example, hydroxy-propyl-β-cyclodextrin or γ-cyclodextrin. In some embodiments, the presence of cyclodextrins allows the use of more concentrated solutions of budesonide.

The budesonide is dissolved can be dissolved in water, alcohol or a water/alcohol mixture.

Exemplary alcohols useful in the preparation of the composition include, but are not limited to, propylene glycol, ethanol or isopropanol.

In embodiments wherein an alcohol/water mixture is employed, the ratio of alcohol to water can be between about 100:0 and 80:20, more preferably between about 98:2 and 93:7.

In some embodiments, the budesonide content in the pharmaceutical composition is between about 0.001 and 1% by weight, between about 0.01 and 0.1% by weight, or between about 0.001 to 0.1% by weight.

In some embodiments, wherein the pharmaceutical composition is provided as a rectal foam, the budesonide content is between about 0.01 and 0.1% by weight.

The pharmaceutical compositions can contain pharmaceutically acceptable excipients known by one of skill in the art to be used in pharmaceutical formulations. For example, such excipients may be suitable for solubilizing corticoids.

Pharmaceutically acceptable excipients include, for example, those which can influence (e.g. increase or decrease) the viscosity of the solution, preservatives (e.g. ethanol, chlorobutanol, benzyl alcohol, phenylethanol, sorbic acid, benzoic acid, sodium disulfite, p-hydroxybenzoates, phenol, m-cresol, p-chloro-m-cresol, quats, chlorohexidine), thickeners (e.g. gelatin, tragacanth, pectin), cellulose derivatives (e.g. methylcellulose, hydroxypropylmethylcellulose, carboxymethylcellulose sodium), polyvinylpyrrolidone, polyvinyl alcohol, polyacrylic acids, xanthan gum, acids (e.g. acetic acid, citric acid, tartaric acid, hydrochloric acid, phosphoric acid), bases (e.g. potassium hydroxide, sodium hydroxide) and buffer substances (e.g. hydrochloric acid buffer, phthalate buffer, phosphate buffer, borate buffer, acetate buffer or citrate buffer). For example, in order to increase the solubility of the active substance, it can be suitable to add sufficient amounts of alcohols (e.g. ethanol, isopropanol, glycerol, propylene glycol, polyethylene glycols) or to use solubilizers (e.g. cyclodextrins, preferably β-cyclodextrin, hydroxypropyl-β-cyclodextrin and/or γ-cyclodextrin).

In embodiments wherein the pharmaceutical composition comprising budesonide is provided as a rectal foam formulation, it may be useful to add pharmaceutically acceptable excipients to assist in forming a dispersion. Excipients may include, for example, emulsifiers, such as Eumulgins and various Lanette types. It is also possible to add preservatives such as, for example, sorbic acid, parahydroxy-benzoates, and acids, such as benzoic acid, acetic acid, citric acid, tartaric acid, hydrochloric acid and phosphoric acid.

Furthermore, in embodiments wherein the pharmaceutical composition is provided as a rectal foam formulation, suitable propellant gases can also introduced into the pressure packs. Suitable propellant gases include, for example, hydrocarbons such as isobutane, n-butane, propane or mixtures thereof.

In some embodiments, the composition comprises sodium EDTA, wherein the sodium EDTA is provided in an amount of about 0.01 to 1.0% by weight of the composition. In some embodiments, wherein the composition is provided as a rectal foam, the composition contains sodium EDTA in an amount of from about 0.05 to 1% by weight.

In some embodiments, the pharmaceutical composition comprises cyclodextrins, wherein the cyclodextrins are provided in an amount of between about 0.05 and 0.5% by weight. In some embodiments, the composition comprises cyclodextrins in an amount of about 0.1% by weight.

In one embodiment, the pharmaceutical composition or formulation comprising budesonide comprises budesonide, propylene glycol, cetyl alcohol, Emulsifying Wax, polyoxyl stearyl ether, Purified Water, Edetate Disodium, citric acid, and nitrogen.

The preparation of solutions for rectal foams may be achieved, for example, by the preservatives and the emulsifiers for the foam formation being dissolved in the appropriate solution, preferably a suitable alcohol. The active substance is then incorporated as alcoholic stock solution into this solution. In the final step, a preservative (e.g. Komplexon) and an appropriate acid, dissolved in a small amount of water, are stirred into the alcoholic solution with homogenization or mixing.

If the budesonide solution is used to produce a rectal foam, the finished solution is, for example, introduced into suitable compressed gas packs which are provided with commercially obtainable valve systems as single or multiple dose devices, and a propellant gas is added. The packs can additionally contain an applicator tip, which, for example, can be made of plastic. Due to the chemical and physical properties of the budesonide solution, the foam, for example, may be generated by the device in the rectum on administration; may be generated in the rectum on administration; may be generated by the device after insertion in the rectum for administration or may be done by any other method known by one of skill in the art.

In some embodiments, the budesonide solutions are used to produce rectal foams. For example, a rectal foam product may be contained in a delivery device. For example, the closure system for the foam product could be comprised of a canister, fitted with (or contained as a part of) a metering valve. The metering valve may further comprise a stem and metering head. The device may further comprise a safety tab to, for example, to prevent accidental actuation. In certain embodiments, the tab could be removed prior to use. The canister may deliver a dose when it is held in any direction, but preferable only when inverted. In certain embodiments, once activated, the valve opens and the metering head dome fills with a single dose of a budesonide foam product emulsion and a propellant mixture. The foam is expelled once the metering head is released. The device may be packaged with bags for safe and hygienic disposal of the used applicators. In a further embodiment, the container closure system for the foam product could be comprised of a 20-100 mL canister, fitted with a 0.25-4-inch metering valve. The valve may be affixed with a 0.20-3 mL metering head. A safety tab may be attached to a foam shield. In certain embodiments, the foam product canister will be provided in a carton containing from 1-4 trays of between 2 and 28 single-use, disposable, rectal applicators.

Propellants for formulation of a foam product can be for example, an alkane in gas form or liquid form, for example, propane, isobutane or butane or mixtures thereof. For example, in one embodiment, the propellant used for the product is a mixture of propane and butane. For example, in one embodiment, the propellant used for the product is a mixture of propane and isobutane. For example, in one embodiment, the propellant used for the product is a mixture of propane, isobutane and butane. In certain embodiments, the propellant is a mixture of propane and butane combined at a molar ratio of 1-25% and 26-99% respectively. In certain embodiments, the propellant is a mixture of propane and isobutane butane combined at a molar ratio of 1-25% and 26-99%, respectively. In certain embodiments, the propellant is a mixture of propane, isobutane and butane combined at a molar ratio of 1-20%, 10-98% and 1-20%, respectively.

In certain embodiments, it is the combination of one or more of the method of treating, the foam formulation, the propellant and the device that leads to the surprising spreading. This leads to the efficacy and surprising safety profile descried herein. This is achieved while decreasing the dosage of the steroid being administered to a subject. The methods described here also meet an unmet medical need.

Budesonide compositions, and methods of making the same, are also described in, for example, U.S. Pat. No. 5,858,998 and U.S. Pat. No. 5,914,122, each of which is incorporated herein by reference in its entirety.

### EXAMPLES

It will be appreciated that the invention should not be construed to be limited to the examples, which are now described; rather, the invention is construed to include any and all applications provided herein and all equivalent variations within the skill of the ordinary artisan.

### Example 1

### Administration of Budesonide for the Treatment of Ulcerative Proctitis or Ulcerative Proctosigmoiditis

Two identical Phase 3, randomized, double-blind, placebo-controlled, multi-center studies were conducted to assess the safety/tolerability profile and clinical efficacy of rectally-administered budesonide foam in subjects who present with active mild to moderate ulcerative colitis, including, ulcerative proctitis or proctosigmoiditis.

A total of 265 subjects in Study 1 were randomized in a 1:1 ratio to receive either 2 mg/25 mL budesonide foam two times per day (BID) for 2 weeks followed by 2 mg/25 mL once daily (QD) for 4 weeks, or placebo foam BID for 2 weeks followed by placebo foam QD for 4 weeks. Additionally, a total of 281 subjects in Study 2 were randomized in a 1:1 ratio to receive either 2 mg/25 mL budesonide foam two times per day (BID) for 2 weeks followed by 2 mg/25 mL once daily (QD) for 4 weeks, or placebo foam BID for 2 weeks followed by placebo foam QD for 4 weeks.

An efficacy endpoint was defined as the proportion of subjects who achieve remission with budesonide foam, as compared to an equivalent volume/regimen of placebo foam administered over 6 weeks (2 mg/25 mL BID for 2 weeks followed by 2 mg/25 mL QD for 4 weeks) in subjects with a diagnosis of active mild to moderate UC, including, UP or UPS. Remission is defined as an endoscopy score of ≦1, a rectal bleeding score of 0, and an improvement or no change from baseline in stool frequency subscales of the Modified Mayo Disease Activity Index (MMDAI) at the end of 6 weeks of treatment or withdrawal.

The Modified Mayo Disease Activity Index (MMDAI) was used to assess the overall disease activity for each subject. The modification made to the original Mayo Index reference (Schroeder et al. 1987. “Coated oral 5-aminosalicylic acid therapy for mildly to moderately active ulcerative colitis.” New Eng J Med 317(26):1625-1629, which is incorporated herein by reference in its entirety) is the deletion of “friability” from an endoscopy score of 1. Therefore, with this modification, the presence of friability reflects an endoscopy score of 2 or 3. The MMDAI evaluates 4 indices each on a scale of 0 to 3 with a maximum total score of 12. The following table summarizes the respective MMDAI subscales for scoring.

TABLE 1
Modified Mayo Disease Activity Index (MMDAI)

| | | | Physician's | Endoscopy/ |
|---|---|---|---|---|
| | Stool | Rectal | Global | Sigmoidoscopy |
| Index | frequency | Bleeding | Assessment | Findings |
| MMDAI or | 0 = Normal | 0 = no | 0 = normal | 0 = normal or |
| Ulcerative | number of | blood seen | 1 = mild | inactive |
| Colitis | stools per | 1 = streaks | disease | disease |
| Symptom | day for | of blood | 2 = | 1 = mild |
| Score | this | with stool | moderate | disease |
| (UCSS) | patient | less than | disease | (erythema, |
| | 1 = 1 to 2 | half the | 3 = severe | decreased |
| | more stools | time | disease | vascular |
| | than normal | 2 = obvious | | pattern) |
| | 2 = 3 to 4 | blood with | | 2 = moderate |
| | more stools | stool most | | disease |
| | than normal | of the time | | (marked |
| | 3 = 5 or | 3 = blood | | erythema, |
| | more stools | alone | | absent |
| | than normal | passed | | vascular |
| | | | | pattern, |
| | | | | friability, |
| | | | | erosions) |
| | | | | 3 = severe |
| | | | | disease |
| | | | | (spontaneous |
| | | | | bleeding, |
| | | | | ulceration) |

Safety endpoints included incidence of treatment-emergent adverse events (AEs) and serious adverse events (SAEs), changes from baseline in clinical laboratory assessments (e.g. urinalysis, hematology, clinical chemistry, cortisol levels), changes from baseline in vital sign assessments, and/or changes from baseline in physical examination findings.

Subject demographics and baseline disease characteristics were similar between placebo and treatment groups. Tables 2-6 show the results of several efficacy endpoints across the placebo and treatment groups in the studies described herein.

TABLE 2
Summary of Efficacy Endpoints - Study 1

| | | Budesonide Foam | |
|---|---|---|---|
| | Placebo | 2 mg/25 mL* | |
| Efficacy Endpoint/Category | (N = 132) | (N = 133) | P-value |
| Achieved Remission - LOCF | 34/132 (25.8%) | 51/133 (38.3%) | 0.0324 |
| Achieved Remission - PP Population | 33/128 (25.8%) | 49/129 (38.0%) | 0.0413 |
| Achieved Remission - Worst Cases | 34/132 (25.8%) | 51/133 (38.3%) | 0.0324 |
| Achieved Remission - Observed Cases | 34/132 (25.8%) | 51/129 (39.5%) | 0.0199 |
| Achieved a MMDAI Rectal Bleeding Score of 0 - | 37/132 (28.0%) | 62/133 (46.6%) | 0.0022 |
| LOCF | | | |
| Achieved a MMDAI Rectal Bleeding Score of 0 - | 36/128 (28.1%) | 59/129 (45.7%) | 0.0042 |
| PP Population | | | |
| Achieved a MMDAI Rectal Bleeding Score of 0 - | 37/132 (28.0%) | 60/133 (45.1%) | 0.0047 |
| Worst Cases | | | |
| Achieved a MMDAI Rectal Bleeding Score of 0 - | 37/132 (28.0%) | 60/128 (46.9%) | 0.0020 |
| Observed Cases | | | |
| Achieved a MMDAI Endoscopy Score of 0 or 1 - | 57/132 (43.2%) | 74/133 (55.6%) | 0.0486 |
| LOCF | | | |
| Achieved a MMDAI Endoscopy Score of 0 or 1 - | 55/128 (43.0%) | 72/129 (55.8%) | 0.04526 |
| PP | | | |
| Achieved a MMDAI Endoscopy Score of 0 or 1 - | 57/132 (43.2%) | 74/133 (55.6%) | 0.0486 |
| Achieved a MMDAI Endoscopy Score of 0 or 1 - | 57/124 (46.0%) | 74/119 (62.2%) | 0.0133 |
| Observed Cases | | | |

Remission is defined as an endoscopy score of 0 or 1, a rectal bleeding score of 0, and an improvement or no change from baseline in stool frequency subscales of the MMDAI at the end of 6 weeks of treatment or withdrawal.

TABLE 3
Summary of Efficacy Endpoints -Study 2

| | | Budesonide Foam | |
|---|---|---|---|
| | Placebo | 2 mg/25 mL* | |
| Efficacy Endpoint/Category | (N = 147) | (N = 134) | P-value |
| Achieved Remission - LOCF | 33/147 (22.4%) | 59/134 (44.0%) | <0.0001 |
| Achieved Remission - PP Population | 33/146 (22.6%) | 57/127 (44.9%) | <0.0001 |
| Achieved Remission - Worst Cases | 33/147 (22.4%) | 59/134 (44.0%) | <0.0001 |
| Achieved Remission - Observed Cases | 33/145 | 59/133 (44.4%) | <0.0001 |
| Achieved a MMDAI Rectal Bleeding Score of 0 - | 42/147 (28.6%) | 67/134 (50.0%) | 0.0002 |
| LOCF | | | |
| Achieved a MMDAI Rectal Bleeding Score of 0 - | 42/146 (28.8%) | 65/127 (51.2%) | 0.0002 |
| PP Population | | | |
| Achieved a MMDAI Rectal Bleeding Score of 0 - | 43/147 (29.3%) | 67/134 (50.0%) | 0.0003 |
| Worst Cases | | | |
| Achieved a MMDAI Rectal Bleeding Score of 0 - | 43/145 (29.7%) | 67/131 (51.1%) | 0.0002 |
| Observed Cases | | | |
| Achieved a MMDAI Endoscopy Score of 0 or 1 - | 54/147 (36.7%) | 75/134 (56.0%) | 0.0013 |
| LOCF | | | |
| Achieved a MMDAI Endoscopy Score of 0 or 1 - | 53/146 | 70/127 (55.1%) | 0.0024 |
| PP | | | |
| Achieved a MMDAI Endoscopy Score of 0 or 1 - | 54/147 | 76/134 (56.7%) | 0.0008 |
| Worst | | | |
| Achieved a MMDAI Endoscopy Score of 0 or 1 - | 54/136 (39.7%) | 76/126 (60.3%) | 0.0009 |
| Observed Cases | | | |

Remission is defined as an endoscopy score of 0 or 1, a rectal bleeding score of 0, and an improvement or no change from baseline in stool frequency subscales of the MMDAI at the end of 6 weeks of treatment or withdrawal.

Study 1 and Study 2 data was pooled to characterize the efficacy of budesonide foam according to the extent of distal disease (UP and UPS). The majority of patients in the pooled population had UPS (n=390; 71.4%) compared with UP (n=153; 28.0%). Demographic and baseline disease characteristics were generally comparable between treatment groups

As shown in Table 4 below, a significantly greater percentage of patients with UP or UPS achieved the primary efficacy outcome of remission at the end of 6 weeks treatment with budesonide foam in the pooled population.

TABLE 4
Summary of Efficacy Endpoints According to the Extent of
Distal Disease (UP and UPS)- Studies 1 and 2 Combined

| | Disease | Budesonide Foam, | | Placebo, | | Treatment | |
|---|---|---|---|---|---|---|---|
| | Extent | n/N (%) | | n/N (%) | | difference, % | P-value |
| Primary | UP | 22/72 | (30.6%) | 13/81 | (16.0%) | 14.6% | 0.0315 |
| efficacy | UPS | 87/193 | (45.1%) | 53/197 | (26.9%) | 18.2% | 0.0002 |
| Rectal | UP | 26/72 | (36.1%) | 16/81 | (19.8%) | 16.3% | 0.0242 |
| bleeding = | UPS | 102/193 | (52.8%) | 62/197 | (31.5%) | 21.3% | <.0001 |
| Endoscopy ≦ 1 | UP | 34/72 | (47.2%) | 34/81 | (42.0%) | 5.2% | 0.4889 |
| | UPS | 113/193 | (58.5%) | 76/197 | (38.6%) | 19.9% | 0.0001 |

TABLE 5
Summary of Efficacy Endpoints Study 2

| | | | Budesonide Foam | | |
|---|---|---|---|---|---|
| | Placebo | | 2 mg/25 mL* | | |
| Efficacy Endpoint/Category | (N = 147) | | (N = 134) | | P-value |
| Number of Weeks Subject | (n = 147) | | (n = 134) | | <0.0001 |
| is a Responder - LOCF | | | | | |
| 0 Weeks | 81 | (55.1%) | 54 | (40.3%) | |
| 1 Weeks | 27 | (18.4%) | 11 | (8.2%) | |
| 2 Weeks | 25 | (17.0%) | 22 | (16.4%) | |
| 3 Weeks | 11 | (7.5%) | 29 | (21.6%) | |
| 4 Weeks | 3 | (2.0%) | 18 | (13.4%) | |

TABLE 6
Summary of Efficacy Endpoints Study 2

| | | Budesonide Foam | |
|---|---|---|---|
| | Placebo | 2 mg/25 mL* | |
| Efficacy Endpoint | (N = 147) | (N = 133) | P-value |
| Achieved a MMDAI Overall Score of <= 2 | 29/147 (19.7%) | 54/134 (40.3%) | 0.0001 |
| Achieved a MMDAI Overall Score of <= 1 | 19/147 (12.9%) | 37/134 (27.6%) | 0.0025 |
| Achieved Improvement of >= 1 Point from | 57/147 (38.8%) | 77/134 (57.5%) | 0.0018 |
| Baseline in the MMDAI Endoscopy Score | | | |
| Achieved Improvement of >= 1 Point from | 83/147 (56.5%) | 97/134 (72.4%) | 0.0058 |
| Baseline in the MMDAI Rectal Bleeding Score | | | |
| Achieved a MMDAI Rectal Bleeding Score of 0 | 35/147 (23.8%) | 62/134 (46.3%) | <0.0001 |
| and a Combined Score of <= 2 for MMDAI | | | |
| Bowel Frequency and Physician's Global | | | |
| Assessment | | | |
| Achieved >= 3 Points Improvement in MMDAI | 50/147 (34.0%) | 72/134 (53.7%) | 0.0008 |
| Total Score Including 1 Point Improvement in | | | |

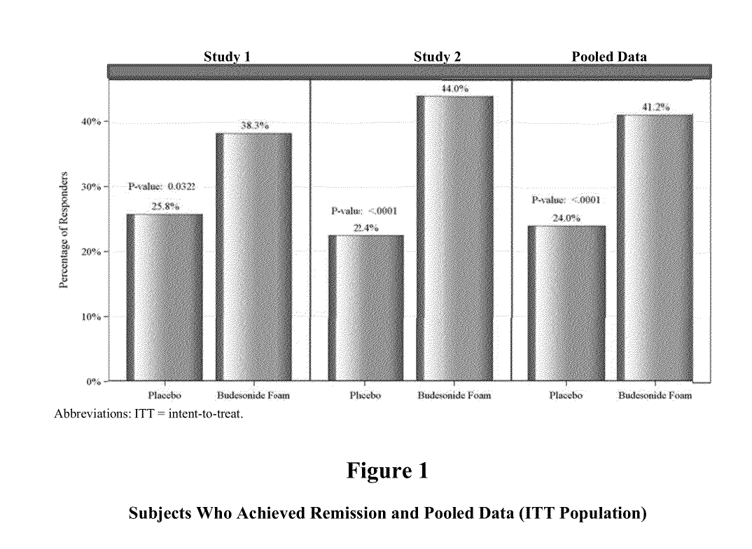

FIG. 1 is a bar chart indicating the percentage of subjects who achieved remission in the treatment and placebo groups in Study 1, Study 2 and combined Study 1 and Study 2 data.

In Study 1, a Phase 3, Randomized, Double-Blind, Placebo-Controlled, Multicenter Study to Assess the Efficacy and Safety of Budesonide Foam (2 mg/25 mL BID for 2 Weeks, Followed by 2 mg/25 mL QD for 4 Weeks) Versus Placebo in Subjects with Active Mild to Moderate Ulcerative Proctitis or Proctosigmoiditis, there were a total of 265 subjects randomized to 1 of 2 double-blind treatment groups: 133 subjects to budesonide 2 mg rectal foam and 132 subjects to placebo.

The rate of combined clinical and endoscopic remission was significantly higher in the budesonide 2 mg rectal foam group (38.3%) compared with the placebo group (25.8%, p=0.0322).

The budesonide foam group achieved higher rates of success than the placebo group for each remission component. Significantly larger proportions of subjects in the budesonide foam group compared with the placebo group achieved an MMDAI endoscopy score of 0 or 1 (budesonide 55.6%, placebo 43.2%; p=0.0488) and an MMDAI rectal bleeding score of 0 (budesonide 46.6%, placebo 28.0%; p=0.0020). The treatment difference in the number of scheduled assessments at which subjects were rectal bleeding responders (achieved a rectal bleeding MMDAI subscale score of 0 during the treatment phase) was statistically significant (p=0.0004) in favor of budesonide. The main response to budesonide was within the first 2 weeks (29.3%), during BID dosing, and this was further improved at Week 4 (47.4%) and maintained at Week 6 (46.6%), during QD dosing.

Significantly larger proportions of subjects in the budesonide foam group compared with the placebo group achieved an MMDAI endoscopy score of 0 or 1 (budesonide 55.6%, placebo 43.2%; p=0.0488) at the end of 6 weeks of treatment.

A significantly larger proportion of subjects in the budesonide foam group (45.9%) compared with the placebo group (30.3%) achieved an MMDAI total score ≦3 with ≧2 point of improvement from baseline at the end of treatment (p=0.0100).

A significantly larger proportion of subjects in the budesonide foam group compared with the placebo group achieved an improvement of ≧1 point from baseline in the MMDAI rectal bleeding subscale score at Weeks 1, 2, 4, and 6 (all post-baseline time points). A significant treatment difference in the proportion of these responders was observed as early as Week 1 (budesonide 57.9%, placebo 40.9%; p=0.0054) and was evident through the final time point at Week 6 (budesonide 70.7%, placebo 53.0%; p=0.0036).

A significantly larger proportion of subjects in the budesonide foam group (52.6%) compared with the placebo group (37.9%) achieved a ≧3 point improvement from baseline in the MMDAI total score, including improvement of ≧1 point from baseline in the MMDAI rectal bleeding subscale score and improvement of ≧1 point from baseline in the MMDAI endoscopy subscale score at the end of 6 weeks of treatment (p=0.0183).

The treatment differences in mean change from baseline to Week 6 were statistically significant (p<0.05) in favor of budesonide for the MMDAI total score and all MMDAI subscale scores (bowel frequency, bleeding, PGA, and endoscopy/sigmoidoscopy findings).

### Study 2

In Study 2, a Phase 3, Randomized, Double-Blind, Placebo-Controlled, Multicenter Study to Assess the Efficacy and Safety of Budesonide Foam (2 mg/25 mL BID for 2 Weeks, Followed by 2 mg/25 mL QD for 4 Weeks) Versus Placebo in Subjects with Active Mild to Moderate Ulcerative Proctitis or Proctosigmoiditis, 281 subjects were randomized to 1 of 2 double-blind treatment groups: 134 subjects to budesonide 2 mg rectal foam and 147 subjects to placebo. Overall, 85% of subjects completed the study (budesonide 86% [115 of 134], placebo 85% [125 of 147]).

A significantly higher in the budesonide foam 2 mg group (44.0%) compared with the placebo group (22.4%, p<0.0001).

In addition, the budesonide foam group achieved higher rates of success than the placebo group for each remission component. Significantly larger proportions of subjects in the budesonide foam group compared with the placebo group achieved an MMDAI endoscopy score of 0 or 1 (budesonide 56.0%, placebo 36.7%; p=0.0012) and an MMDAI rectal bleeding score of 0 (budesonide 50.0%, placebo 28.6%; p=0.0001). A numerically larger proportion of subjects in the budesonide foam group (79.9%) achieved improvement or no change from baseline in the MMDAI bowel frequency score compared with the placebo group (72.8%).

A significantly larger proportions of subjects in the budesonide foam group compared with the placebo group achieved an MMDAI rectal bleeding score of 0 (budesonide 50.0%, placebo 28.6%; p=0.0001) at the end of 6 weeks of treatment.

The treatment difference in the number of scheduled assessments that subjects were rectal bleeding responders (achieved a rectal bleeding MMDAI subscale score of 0 during the treatment phase) was statistically significant (p<0.0001) in favor of budesonide. The main response to budesonide was within the first 2 weeks (41.8%), during BID dosing, and the effect was improved at Week 4 (48.5%) and maintained at Week 6 (50.0%) during QD dosing.

A significantly larger proportions of subjects in the budesonide foam group compared with the placebo group achieved an MMDAI endoscopy score of 0 or 1 (budesonide 56.0%, placebo 36.7%; p=0.0012) at the end of 6 weeks of treatment.

A significantly larger proportion of subjects in the budesonide foam group compared with the placebo group achieved a score of 0 for rectal bleeding subscale and a combined score of ≦2 for bowel frequency and PGA in the MMDAI subscales at Weeks 1, 2, 4, and 6 (all post-baseline time points). A substantial treatment difference in the proportion of these responders was observed as early as Week 1 (budesonide 14.9%, placebo 6.1%; p=0.0160) and was evident through the final time point at Week 6 (budesonide 46.3%, placebo 23.8%; p<0.0001).

A significantly larger proportion of subjects in the budesonide foam group (49.3%) compared with the placebo group (28.6%) achieved an MMDAI total score ≦3 with ≧2 points of improvement at the end of treatment (p=0.0003).

A significantly larger proportion of subjects in the budesonide foam group (57.5%) compared with the placebo group (38.8%) achieved improvement of ≧1 point from baseline in the MMDAI endoscopy subscale score at the end of treatment (p=0.0017).

A significantly larger proportion of subjects in the budesonide foam group compared with the placebo group achieved an improvement of ≧1 point from baseline in the MMDAI rectal bleeding subscale score at Weeks 1, 2, 4, and 6 (all post-baseline time points). A significant treatment difference in the proportion of these responders was observed as early as Week 1 (budesonide 73.1%, placebo 48.3%; p<0.0001) and was evident through the final time point at Week 6 (budesonide 72.4%, placebo 56.5%; p=0.0056).

A significantly larger proportion of subjects in the budesonide foam group (53.7%) compared with the placebo group (34.0%) achieved a ≧3 point improvement from baseline in the MMDAI total score, including improvement of ≧1 point from baseline in the MMDAI rectal bleeding subscale and improvement of ≧1 point from baseline in MMDAI endoscopy subscale, at the end of 6 weeks of treatment (p=0.0007).

The treatment differences in mean change from baseline to Week 6 were statistically significant (p<0.05) in favor of budesonide for the MMDAI total score and all MMDAI subscale scores (bowel frequency, bleeding, PGA, and endoscopy/sigmoidoscopy findings).

The results indicate that the budesonide treatment group exhibited significantly better results than the placebo population with respect to achieving remission of disease, achieving an improved MMDAI rectal bleeding score and achieving an improved MMDAI endoscopy score. Furthermore, the differences between treatment groups became more significant as treatment duration progressed, with stark improvement between treatment outcomes observed at four weeks after the subjects began receiving treatment.

### Example 2

### Safety Profile of Budesonide in the Treatment of Ulcerative Proctitis or Ulcerative Proctosigmoiditis

In subjects with mild to moderate distal Ulcerative Colitis, rectally administered budesonide foam was generally well tolerated, associated with a low incidence of AEs, and did not adversely affect the hypothalamic-pituitary-adrenal axis.

As described above, two identically designed, randomized, double-blind, placebo-controlled, phase 3 studies were conducted. Safety assessments were performed, including monitoring of adverse events and clinical laboratory parameters, such as morning cortisol concentrations and adrenocorticotropic hormone (ACTH) challenge tests. Blood samples for budesonide pharmacokinetics were collected at randomization and weeks 1, 2, 4, and 6.

Results concluded that budesonide foam was generally well tolerated, with the majority of reported adverse events being mild to moderate in intensity (Table 7)

Glucocorticoid adverse effects reported as AEs, such as moon face, striae rubrae, flushing, fluid retention, mood changes, sleep changes, insomnia, acne, and hirsutism, were infrequently reported (<2%); in addition, patients with AEs of adrenal insufficiency or patients with abnormal ACTH challenge results did not report clinical signs and symptoms associated with adrenal suppression. No deaths occurred during the studies.

TABLE 7
Summary of Adverse Events (pooled safety population)

| Adverse | | Budesonide foam | | | |
|---|---|---|---|---|---|
| event, | | 2 mg/25 mL | | Placebo | |
| n (%) | | (n = 268) | | (n = 278) | |
| Any AE | | 123 | (45.9) | 101 | (36.3) |
| | Drug-related AEs | 56 | (20.9) | 16 | (5.8) |
| | Discontinuations | 26 | (9.7) | 12 | (4.3) |
| | due to AE | | | | |
| | Serious AEs | 5 | (1.9) | 3 | (1.1) |
| Intensity | Mild | 88 | (32.8) | 57 | (20.5) |
| of AE | Moderate | 27 | (10.1) | 40 | (14.4) |
| | Severe | 8 | (3.0) | 4 | (1.4) |
| Most | Decreased blood | 46 | (17.2) | 6 | (2.2) |
| common | cortisol levels | | | | |
| AEs | Adrenal insufficiency^{d} | 10 | (3.7) | 2 | (0.7) |
| | Headache | 6 | (2.2) | 7 | (2.5) |
| | Nausea | 6 | (2.2) | 2 | (0.7) |

| | | | |
|---|---|---|---|
| Ulcerative proctitis | 0 | 6 | (2.2) |

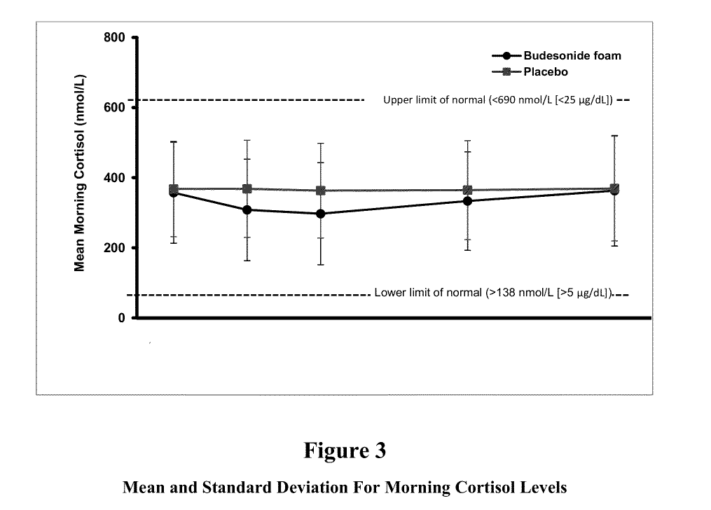

Further, the mean morning cortisol level concentrations remained within normal levels following treatment with budesonide foam. Although a transient decrease in mean cortisol concentrations was observed during twice-daily dosing, a recovery to baseline concentrations was observed by week 6 (FIG. 3). The majority of patients treated with budesonide foam maintained normal total cortisol concentrations (>138 nmol/L) and normal responses to the ACTH challenge (Table 8).

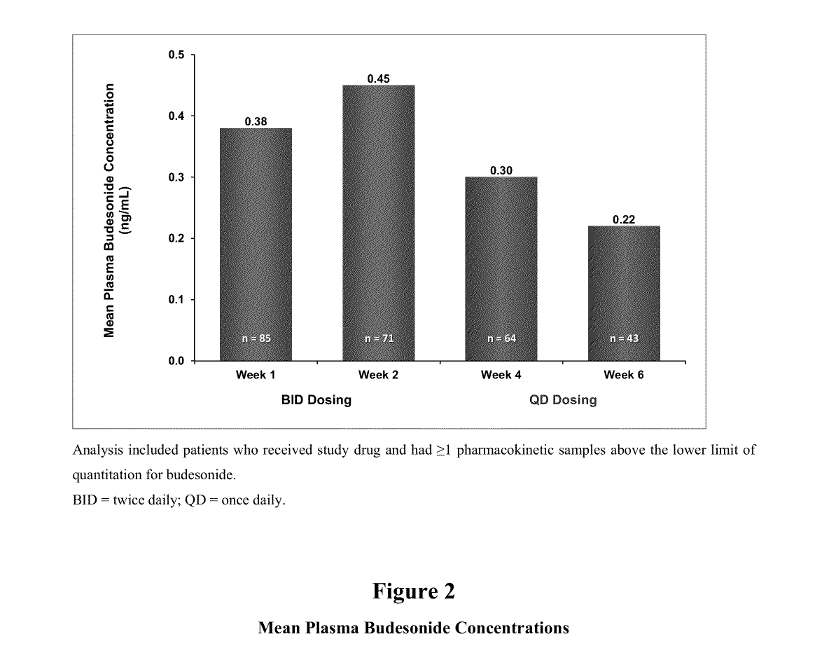

Additionally, low systemic exposure of budesonide was observed, with 33% of samples below the level of quantitation (0.03 ng/mL). For samples above the limit of quantitation, mean plasma budesonide concentrations were greater during twice-daily dosing compared with once-daily dosing, but overall there was low systemic bioavailability observed (FIG. 2).

Budesonide systemic exposure (AUC and Cmax) did not correlate with decreased sensitivity to ACTH challenge at week 6, suggesting that budesonide foam did not have any apparent clinically relevant effects on the HPA axis. The P value for the slope of the AUC relationship to percentage change in cortisol levels was <0.05, which suggested that an increase in budesonide exposure predicts an increase in responsiveness of the HPA axis, as measured by the ACTH stimulation test; this positive correlation was the opposite of what would be expected if increases in budesonide systemic exposure resulted in a decrease in responsiveness of the HPA axis.

TABLE 8
Percentage of Patients With Total Cortisol Levels >5 μg/dL and
Percentage of Patients With Normal Response to ACTH Challenge

| | | Budesonide Foam | |
|---|---|---|---|
| | | 2 mg/25 mL | Placebo |
| Parameter, n/N^{a} (%) | | (n = 268) | (n = 278) |
| Total | Baseline | 259/268 (96.6) | 275/278 (98.9) |
| cortisol >5 μg/dL | Week 1 (BID) | 224/263 (85.2) | 264/269 (98.1) |
| (138 nmol/L) | Week 2 (BID) | 216/257 (84.0) | 263/266 (98.9) |
| | Week 4 (QD) | 218/235 (92.8) | 243/249 (97.6) |
| | Week 6 (QD) | 211/224 (94.2) | 234/241 (97.1) |
| Normal response to | Baseline | 222/266 (83.5) | 238/278 (85.6) |
| ACTH challenge^{b} | Week 6 | 148/216 (68.5) | 180/235 (76.6) |

^{a}Denominator N is the number of patients with a value at each given week during the study.
^{b}The normal response to ACTH challenge includes 3 criteria, as defined in the cosyntropin label: 1) morning cortisol level >5 μg/dL (pre-challenge; 138 nmol/L); 2) increase in cortisol level by ≧7 μg/dL (193 nmol/L) above the morning (pre-challenge) level following ACTH challenge; and 3) cortisol level of >18 μg/dL (500 nmol/L) following ACTH challenge.
ACTH = adrenocorticotropic hormone; BID = twice daily; QD = once daily.

### Example 3

### Analysis of Adverse Events

Tables 9-11 provide a summary of treatment-emergent adverse events in the study.

TABLE 9
Treatment-Emergent Adverse Events by System - Study 1
Study 1 and Study 2 Combined Data: The most frequently reported TEAEs
by preferred term (in ≧3% of subjects in the budesonide foam or
placebo group) were blood cortisol decreased (budesonide 17%, placebo
2%), adrenal insufficiency (budesonide 4%, placebo 0.7%), and
headache (budesonide 2%, placebo 3%). (Table 9-11)

| | | Budesonide Foam |
|---|---|---|
| | Placebo | 2 mg/25 mL |
| | (N = 147) | (N = 134) |
| System Organ Class | n(%) | n(%) |
| Respiratory, thoracic and | 2 (1.5%) | 0 |
| mediastinal disorders | | |
| Gastrointestinal disorders | 0 | 2 (1.5%) |
| Headache | 1 (0.8%) | 4 (3%) |

TABLE 10
Treatment-Emergent Adverse Events by System - Study 2

| | | Budesonide Foam |
|---|---|---|
| | Placebo | 2 mg/25 mL |
| | (N = 147) | (N = 134) |
| System Organ Class | n(%) | n(%) |
| Respiratory, thoracic and | 1 (0.7%) | 2(1.5%) |
| mediastinal disorders | | |
| Gastrointestinal disorders | 0 | 2(1.5%) |
| Headache | 6 (4.1%) | 2(1.5%) |

Study 1 and Study 2 Combined Data demonstrate that the most frequently reported TEAEs by preferred term (in ≧3% of subjects in the budesonide foam or placebo group) were blood cortisol decreased (budesonide 17%, placebo 2%), adrenal insufficiency (budesonide 4%, placebo 0.7%), and headache (budesonide 2%, placebo 3%). This is surprising and advantageous over previous methods of using a budesonide foam product. For example, when subjects administered 2 or 4 mg budesonide foam BID for 6 weeks or 2 mg QD for 8 weeks the incidence of headache were between 3% and 10.1%.

As seen in the novel studies and methods described herein, a significant difference in headache (fewer headaches) were experienced in this method, thus reducing a significant deterrent for subjects to comply with treatment.

In certain embodiments, subjects being administered a foam composition comprising budesonide 2 mg budesonide BID for two weeks, followed by 2 mg budesonide QD for four weeks, experience headaches in about 2% of the subjects. In certain embodiments, subjects being administered a foam composition comprising budesonide 2 mg budesonide BID for two weeks, followed by 2 mg budesonide QD for four weeks, experience headaches in about 1.5% of the subjects. In certain embodiments, subjects being administered a foam composition comprising budesonide 2 mg budesonide BID for two weeks, followed by 2 mg budesonide QD for four weeks, experience headaches in below 3% of the subjects. In certain embodiments, subjects being administered a foam composition comprising budesonide 2 mg budesonide BID for two weeks, followed by 2 mg budesonide QD for four weeks, experience headaches in between about 1.5-3% of the subjects.

The presently described methods are also advantageous because they case fewer respiratory incidents. In Study 1 and Study 2, 0% of the subjects experienced respiratory adverse events. In previous methods, for example, when subjects administered 2 or 4 mg budesonide foam BID for 6 weeks or 2 mg QD for 8 weeks the incidence of respiratory adverse events were about 3%. In certain embodiments, subjects being administered a foam composition comprising budesonide 2 mg budesonide BID for two weeks, followed by 2 mg budesonide QD for four weeks, experience respiratory adverse events in about 0% of the subjects.

In further support of the improved safety of these methods, Study 1 and Study 2 data were pooled and the safety data was analyzed for two populations of subject, UC and UPS. See Table 11 for the results.

TABLE 11
Safety. Summary of Adverse Events (Study 1 and Study 2, pooled data -
According to the extent of distal disease (UP and UPS)
The safety profile of budesonide foam was comparable between patients
with UP and UPS, with most AEs mild or moderate in intensity

| | | Ulcerative | | | | Ulcerative | | | |
|---|---|---|---|---|---|---|---|---|---|
| | | Proctitis | | | | Proctosigmoiditis | | | |
| | | Budesonide | | | | Budesonide | | | |
| | | Foam | | | | Foam | | | |
| | | 2 mg/25 mL | | Placebo | | 2 mg/25 mL | | Placebo | |
| Adverse event, (%) | | (n = 72) | | (n = 81) | | (n = 194) | | (n = 196) | |
| Any AE | Drug-related AEs | 36 | (50.0) | 31 | (38.3) | 87 | (44.8) | 70 | (35.7) |
| | Discontinuations due to AE | 1.4 | (19.4) | 4 | (4.9) | 42 | (21.6) | 12 | (6.1) |
| | Serious AEs | 7 | (9.7) | 3 | (3.7) | 19 | (9.8) | 9 | (4.6) |
| | | 1 | (1.4) | 0 | | 4 | (2.1) | 3 | (1.5) |
| Intensity | Mild | 23 | (31.9) | 20 | (24.7) | 65 | (33.5) | 37 | (18.9) |
| of AE^{a} | Moderate | 10 | (13.9) | 9 | (11.1) | 17 | (8.8) | 31 | (15.8) |
| | Severe | 3 | (4.2) | 2 | (2.5) | 5 | (2.6) | 2 | (1.0) |
| Most | Decreased blood cortisol | 10 | (13.9) | 0 | | 36 | (18.6) | 6 | (3.1) |
| common | Nausea | 4 | (5.6) | 0 | | 2 | (1.0) | 2 | (1.0) |
| AEs^{b} | Adrenal insufficiency | 3 | (4.2) | 1 | (1.2) | 7 | (3.6) | 1 | (0.5) |
| | Diarrhea | 2 | (2.8) | 0 | | — | | — | |
| | Back pain | 2 | (2.8) | 2 | (2.5) | 1 | (0.5) | 1 | (0.5) |
| | Headache | 1 | (1.4) | 0 | | 5 | (2.6) | 7 | (3.6) |
| | Ulcerative colitis | 0 | | 1 | (1.2) | 3 | (1.5) | 4 | (2.0) |
| | Arthralgia | 1 | (1.4) | 2 | (2.5) | 1 | (0.5) | 2 | (1.0) |
| | Urinary tract infection | 1 | (1.4) | 3 | (3.7) | 2 | (1.0) | 2 | (1.0) |

| Increased blood bilirubin | 0 | 3 | (3.7) | 0 | | 2 | (1.0) |
|---|---|---|---|---|---|---|---|
| Anemia | — | — | | 1 | (0.5) | 5 | (2.6) |
| Abdominal tenderness | 0 | 2 | (2.5) | 1 | (0.5) | 2 | (1.0) |
| Ulcerative proctitis | 0 | 1 | (1.2) | 0 | | 5 | (2.6) |
| Hemorrhoids | 0 | 2 | (2.5) | 0 | | 2 | (1.0) |
| Oropharyngeal pain | 0 | 2 | (2.5) | 0 | | 1 | (0.5) |
| Increased AST | 0 | 2 | (2.5) | 3 | (1.5) | 2 | (1.0) |

^{a}Patients experiencing ≧1 AE are counted once and categorized by the intensity of the most severe AE.
^{b}AEs include ≧2% of patients in any treatment group.
AE = adverse event; AST = aspartate aminotransferase.

### Example 4

### Budesonide Plasma Concentrations

Blood samples for population pharmacokinetic analysis of plasma budesonide concentrations were collected from 125 subjects (60 placebo, 65 budesonide rectal foam, Table 12). During the twice-daily administration phase in Weeks 1 and 2, mean plasma budesonide concentrations in samples above the limit of quantitation were higher (0.367 and 0.422 ng/mL in Weeks 1 and 2, respectively) than during once-daily administration (Weeks 4 and 6, 0.244 and 0.184 ng/mL, respectively). The highest plasma concentration observed in a budesonide-treated subject was 2.22 ng/mL. In placebo-treated subjects, 1 of 253 post-randomization samples collected had a quantifiable budesonide concentration (2.46 ng/mL.) Examination of dosing records, concomitant medications, and bioanalytical records reveals no source of dosing error, assay cross-reactivity, or analytical error.

Assessment of concentration-time data demonstrates that maximum plasma concentrations of budesonide in subjects with UP or UPS are similar to those observed in healthy subjects receiving budesonide rectal foam. In addition, systemic exposure of budesonide is similar to the systemic exposures reported for an extended-release oral formulation of budesonide that was recently approved by the US FDA for the induction of remission of UC. However, budesonide MMX had a substantially higher steady-state AUC compared with budesonide rectal foam (16.43 versus 4.30 ng.h/mL, respectively.) Further, results indicated that the systemic exposure of budesonide foam is affected by the severity of the disease state. The study revealed that there is a decrease in the elimination rate constant associated with an increase in the severity of disease symptoms.

TABLE 12
Budesonide Plasma Concentrations Study 1

| | Placebo | Budesonide 2 mg |
|---|---|---|
| Randomization^{b} | n = 53 | n = 55 |
| Plasma budesonide ≧ LLQ (≧0.03 ng/mL)^{c} | n = 0 | n = 1 |
| Plasma budesonide concentration, mean ± SD, ng/mL | — | 0.093 |
| Week 1^{b} | n = 51 | n = 59 |
| Plasma budesonide ≧ LLQ (≧0.03 ng/mL)^{c} | n = 0 | n = 45 |
| Plasma budesonide concentration, mean ± SD | — | 0.367 ± 0.4290 |
| Week 2^{b} | n = 47 | n = 50 |
| Plasma budesonide ≧ LLQ (≧0.03 ng/mL)^{c} | n = 1 | n = 37 |
| Plasma budesonide concentration, mean ± SD | — | 0.422 ± 0.4546 |
| Week 4^{b} | n = 47 | n = 50 |
| Plasma budesonide ≧ LLQ (≧0.03 ng/mL)^{c} | n = 0 | n = 32 |
| Plasma budesonide concentration, mean ± SD | — | 0.244 ± 0.2532 |
| Week 6^{b} | n = 49 | n = 55 |
| Plasma budesonide ≧ LLQ (≧0.03 ng/mL)^{c} | n = 0 | n = 17 |
| Plasma budesonide concentration, mean ± SD | — | 0.184 ± 0.2632 |

a Number of subjects, N, equals the number who received study medication and had ≧1 sample that was analyzed for budesonide concentrations.
^{b}Number of subjects, n, equals the number who had samples at that time point that were assayed for determination of plasma budesonide concentrations.
^{c}Number of subjects, n, equals the number who had plasma budesonide concentrations ≧ LLQ (≧0.03 ng/mL).

The methods described herein increase the safety and compliance of the use of budesonide foam over previous methods. The methods of treatment and dosing schedules provided herein are advantageous over other methods and dosing schedules due to the safety profile of this drug. Based on the data shown in Table 9 as compared with previous budesonide products, headaches are reduced over previous dosing regimes by 50-70% (from 6% to 1.5% of the study participants). Nervous system disorders are reduced by 66% (from 10.9% to 3.7%). Gastrointestinal side effects were reduced by 87% (from 11.6% to 1.5%). Respiratory complications were reduced by 32-50% (from 3-2.2% to 1.5%). These increases in safety and side effect profiles will increase patient compliance. Thus, the instant budesonide dosing and treatment regimen were considered to be safe as well as effective in treating subjects with mild to moderate active UC, including UP and/or UPS. The comparisons described above were relative to a budesonide foam administered either as 2 mg QD for 8 weeks or 2 mg or 4 mg BID for 6 weeks. Surprisingly, budesonide foam administered with less drug load over the course of treatment or decrease in the exposure time over the course of treatment led to a reduction in the side effects and in greater efficacy.

### Example 5

### Delivery Device

Budesonide 2 mg Rectal Foam is an aerosol foam delivered by a disposable, non-priming, dosemetering, multi-dose canister. The drug product formulation is a non-sterile emulsion consisting of budesonide, propylene glycol, cetyl alcohol, emulsifying wax, polyoxyl (10) stearyl ether, purified water, edetate disodium, and citric acid monohydrate. The emulsion is filled into a 54-mL, white, aluminum monoblock canister coated internally with protective epoxyphenolic resins. Each canister is fitted with a 1-inch metered-valve system consisting of a polyester valve body and stem. A propellant consisting of propane, isobutane, and butane is added to the crimp-sealed can before a 1.35-mL dispenser head and a polypropylene foam shield are installed. Each multi-dose canister delivers fourteen 1.35-mL doses of foam product (equivalent to 2 mg budesonide per dose) and will be provided with 14 single-use, disposable, white, polyvinyl chloride rectal applicators. Each applicator is pre-coated with paraffin lubricant and stored in a protective, white, low density polyethylene tray (7 applicators per tray). Plastic bags are included in the secondary packaging for safe and hygienic disposal of the used applicators.

Prior to the first dose, the “safety tab” provided on the foam shield of the canister will be removed by the user. After shaking the canister, the user will attach an applicator to the delivery nozzle of the dosing valve, invert the canister and depress the pump dome. The user will then insert the applicator into the rectum and release the pump dome to deliver the foam product. After delivery of the foam, the user will remove the applicator and place it in a plastic disposal bag. A new applicator will be used for each dose.

### INCORPORATION BY REFERENCE

The contents of all references, patents, pending patent applications and published patents, cited throughout this application are hereby expressly incorporated by reference.

### EQUIVALENTS

Those skilled in the art will recognize, or be able to ascertain using no more than routine experimentation, many equivalents to the specific embodiments of the invention described herein. Such equivalents are intended to be encompassed by the following claims.

What is claimed is:

1. A method of treating ulcerative colitis (UC) in a subject, comprising administering a foam composition comprising budesonide to the subject, wherein the subject is administered 2 mg budesonide BID for two weeks, followed by 2 mg budesonide QD for four weeks.

2. The method of claim 1, wherein the composition is administered rectally.

3. The method of claim 1, wherein the subject suffers from active mild to moderate ulcerative proctitis and/or ulcerative proctosigmoidits.

4. The method of claim 1, wherein the subject suffers from at least one symptom selected from the group of: rectal bleeding, urgency, tenesmus, diarrhea, constipation and rectal pain.

5. The method of claim 1, wherein the disease extends from about 5 cm to about 40 cm from the anal verge of the subject.

6. The method of claim 5, wherein the disease extends about 15 cm from the anal verge of the subject.

7. The method of claim 1, wherein the disease extends up to about 40 cm from the anal verge of the subject.

8. The method of claim 1, wherein the subject exhibits histological changes characteristic of ulcerative colitis, ulcerative proctitis and/or ulcerative proctosigmoidits.

9. The method of claim 1, wherein the subject exhibits a Modified Mayo Disease Activity Index (MMDAI) score of between about 5 and 10 prior to administration of the composition.

10. The method of claim 1, wherein the subject exhibits a score of ≧2 on the MMDAI rectal bleeding component prior to administration of the composition.

11. The method of claim 1, wherein the subject exhibits a score of ≧2 on the MMDAI endoscopy or sigmoidoscopy component prior to administration of the composition.

12. The method of claim 1, wherein administration of the composition results in at least one selected from the group of: an endoscopy score of ≦1, a rectal bleeding score of 0, and an improvement or no change from baseline in stool frequency subscales of the Modified Mayo Disease Activity Index (MMDAI).

13. The method of claim 1, wherein administration of the composition results in at least one selected from the group of: an endoscopy score of ≦1, a rectal bleeding score of 0, and an improvement or no change from baseline in stool frequency subscales of the Modified Mayo Disease Activity Index (MMDAI).

14. The method of claim 13, wherein an improvement in stool frequency comprises a combined score of ≦2 for bowel frequency and physician's global assessment in the MMDAI subscales.

15. The method of claim 1, wherein administration of the composition results in an MMDAI rectal bleeding score of 0.

16. The method of claim 1, wherein administration of the composition results in an MMDAI endoscopy score of 0 or 1.

17. The method of claim 1, wherein administration of the composition results in an MMDAI overall of ≦2.

18. The method of claim 17, wherein administration of the composition results in an MMDAI overall of ≦1.

19. The method of claim 1, wherein administration of the composition results in an improvement of ≧1 point from baseline in the MMDAI endoscopy score.

20. The method of claim 1, wherein administration of the composition results in an improvement of ≧1 point from baseline in the MMDAI rectal bleeding score.

21. The method of claim 1, wherein administration of the composition results in an improvement of ≧3 points from baseline in the MMDAI total score, including a 1 point improvement in both rectal bleeding and endoscopy scores.

22. The method of claim 1, wherein an improvement in disease symptoms and/or progress is observed for up to 6 weeks after administration of the composition commences.

23. The method of claim 1, wherein an improvement in disease symptoms and/or progress is observed for up to 4 weeks after administration of the composition commences.

24. The method of claim 1, wherein incidence of headaches is lower than in subjects administered 2 or 4 mg budesonide foam BID for 6 weeks or 2 mg QD for 8 weeks.

25. The method of claim 1, wherein incidence of nervous system disorders is lower than in subjects administered budesonide foam 2 mg QD for 8 weeks.

26. The method of claim 1, wherein incidence of nervous system disorders is lower than in subjects administered budesonide foam 2 mg QD for 4 weeks.

27. The method of claim 1, wherein incidence of respiratory side effect is lower than in subjects administered budesonide foam 2 or 4 mg budesonide foam BID for 6 weeks or 2 mg QD for 4 or 8 weeks.

28. The method of claim 1, wherein incidence of gastrointestinal side effects is lower than in subjects administered budesonide foam 2 mg QD for 4 weeks.

29. A method of alleviating symptoms in a subject with ulcerative colitis, comprising administering a foam composition comprising budesonide to the subject, wherein the subject is administered 2 mg budesonide BID for two weeks, followed by 2 mg budesonide QD for four weeks.

30. The method of claim 29, wherein the symptoms are selected from the group consisting of diarrhea, constipation, urgency, tenesmus, rectal bleeding, rectal pain, cramping and abdominal pain.

31. A method of treating ulcerative colitis, comprising administering a foam composition comprising budesonide to the subject, wherein the subject is administered 2 mg budesonide BID for two weeks, followed by 2 mg budesonide QD for four weeks, wherein subjects experience a lower than expected systemic level of budesonide in the four weeks of QD dosing.

32. The method of any of claim 1 or 31, wherein, an increased age in a subject correlates to a decrease in the systemic elimination rate of budesonide.

33. The method of claim 29, wherein an increase in the severity of the disease state in a patient correlates to a decrease in the elimination rate of budesonide.

34. The method of claim 33, wherein the severity of the disease state is measured by the MMDAI.

35. The method of claim 33, wherein the severity of the disease state is determined by the state of the patient's endoscopic disease.

36. The method of claim 33, wherein the severity of the disease state is determined by total disease severity.

37. The method of claim 1, wherein the incidence of headaches in the subjects is at about 2%.

38. The method of claim 1, wherein the incidence of headaches in the subjects is at about 1.5% of the subjects.

39. The method of claim 1, wherein the incidence of headaches in the subjects is below 3%.

40. The method of claim 1, wherein the incidence of headaches in the subjects is in between about 1.5-3%.

41. The method of claim 1, wherein the incidence of respiratory adverse events in the subjects occurs in about 0% of the subjects.

42. The method of claim 1, wherein the foam composition is administered with a device comprising a canister and a metering valve.

43. The method of claim 42 wherein the metering valve further comprises a stem.

44. The method of claim 42, wherein the device further comprises a safety tab.

45. The method of claim 44, wherein the safety tab prevents accidental actuation.

46. The method of claim 42, wherein the device delivers a dose only when inverted.

47. A method of inducing remission in subjects with active mild to moderate distal ulcerative colitis (UC) comprising, administering a foam composition comprising budesonide to the subject, wherein the subject is administered 2 mg budesonide twice daily (BID) for two weeks, followed by 2 mg budesonide once daily (QD) for four weeks.

48. The method of claim 47, wherein the ulcerative colitis extends from about 1 cm to about 5 cm from the anal verge of the subject.

49. The method of claim 47, wherein the ulcerative colitis extends from about 5 cm to about 40 cm from the anal verge of the subject.

50. The method of claim 47, wherein the ulcerative colitis extends about 15 cm from the anal verge of the subject.

51. The method of claim 47, wherein the ulcerative colitis extends up to about 40 cm from the anal verge of the subject.

52. A method of inducing remission in subjects with active mild to moderate distal ulcerative colitis (UC) extending up to 40 cm from the anal verge comprising administering a foam composition comprising budesonide to the subject, wherein the subject is administered 2 mg budesonide twice daily (BID) for two weeks, followed by 2 mg budesonide once daily (QD) for four weeks.

53. A method of inducing remission in a subject with ulcerative proctitis, comprising administering a foam composition comprising budesonide to the subject, wherein the subject is administered 2 mg budesonide twice daily (BID) for two weeks, followed by 2 mg budesonide once daily (QD) for four weeks.

54. The method of claim 53, wherein administration of the composition results in at least one selected from the group of: an endoscopy score of ≦1, a rectal bleeding score of 0, and an improvement or no change from baseline in stool frequency subscales of the Modified Mayo Disease Activity Index (MMDAI).

55. The method of claim 53, wherein incidence of headaches is lower than in subjects administered 2 or 4 mg budesonide foam BID for 6 weeks or 2 mg QD for 8 weeks.

56. A method of inducing remission in a subject with ulcerative proctosigmoiditis, comprising administering a foam composition comprising budesonide to the subject, wherein the subject is administered 2 mg budesonide twice daily (BID) for two weeks, followed by 2 mg budesonide once daily (QD) for four weeks.

57. The method of claim 56, wherein administration of the composition results in at least one selected from the group of: an endoscopy score of ≦1, a rectal bleeding score of 0, and an improvement or no change from baseline in stool frequency subscales of the Modified Mayo Disease Activity Index (MMDAI).

58. The method of claim 57, wherein incidence of headaches is lower than in subjects administered 2 or 4 mg budesonide foam BID for 6 weeks or 2 mg QD for 8 weeks.
